(19) United States
(12) Patent Application Publication
(10) Pub. No.: US 2015/0272517 A1
(43) Pub. Date: Oct. 1, 2015

(54) SCALABLE AND FLEXIBLE CT DETECTOR HARDWARE TOPOLOGY

(71) Applicant: Siemens Aktiengesellschaft, Muenchen (DE)

(72) Inventors: Alex GLEICH, Fuerth (DE); Alexander GRAF, Forchheim (DE); Sven GÜNTHER, Goessweinstein (DE); Arne HARRIERS, Uttenreuth (DE); Erhard SCHLUND, Wiesenthau (DE); Matthias SCHMIDT, Moehrendorf (DE); Andreas WESTERKOWSKY, Nuernberg (DE); Klaus WINDSHEIMER, Spalt (DE)

(21) Appl. No.: 14/666,741

(22) Filed: Mar. 24, 2015

(30) Foreign Application Priority Data
Mar. 31, 2014 (DE) 102014206007.9

(51) Int. Cl. A61B 6/03; A61B 6/00

(52) U.S. Cl. CPC A61B 6/035; A61B 6/54; A61B 6/56

(57) ABSTRACT

An imaging device is described. The imaging device includes a central communications unit, at least one individual detector control unit, a plurality of individual detectors and a serial interface between the at least one individual detector control unit and the central communications unit. Furthermore, a method for manufacturing an imaging device is described. In the method, a central communications unit is arranged between at least one individual detector control unit and a control and image data transmission unit. In addition, a serial interface is formed between the at least one individual detector control unit and the central communications unit.

### PRIORITY STATEMENT

The present application hereby claims priority under 35 U.S.C. §119 to German patent application number DE 102014206007.9 filed Mar. 31, 2014, the entire contents of which are hereby incorporated herein by reference.

### FIELD

At least one embodiment of the invention relates to a medical imaging device and a method for manufacturing the medical imaging device.

### BACKGROUND

Medical imaging devices, in particular conventional computed tomography systems, are synchronized to a specific application with respect to the hardware structure, which leads to a limited flexibility in the construction. An attempt is generally made to connect as many as possible individual detectors via as few as possible flat panels. Between the flat panels, large connectors with many parallel contacts are used as connection terminals in order to run as many communications interfaces as possible in parallel from and to the detectors and the periphery.

A central flat control panel is also often used, which controls communication not only with the individual detectors, but also with the other flat panels. Through this type of control originating from one of the flat panels, an individual solution that matches a specific application is achieved. However, this solution can only be adapted with difficulty to changing conditions and it is also a disadvantage with regard to troubleshooting and to upscaling the arrangement. Multiple use of the individual flat panels is rarely possible.

In a conventional medical technology imaging system, for example in a computed tomography system (see FIG. 1), the various data, the control data, the image data and the status data is usually transmitted via separate interfaces, (see data lines in FIG. 1). A CT detector can consist, for example, of 46 electronics modules. All the electronics modules have to be configured and controlled. The image data and status information therefrom have to be sorted, pre-processed and transmitted to the host PC. An interface for the transmission of image, status and control data is conventionally required between the different function blocks in the system. At the same time, the predetermined geometry, the number of individual detectors, the detector data rate and the functionality requirements have to be considered.

As already mentioned, in the conventional system, the transmission of control data and image data and status information are largely separated from one another (see FIG. 1). There are many different data connections separate from one another, which always have only one purpose and which are routed through the entire system hierarchy. Only limited functions that make it possible to observe the entire system are incorporated into the existing architecture. Only rigid connections exist between the individual function blocks. The connections required between the function blocks are firmly established in the architecture. Each communications interface is present throughout the entire hierarchy, and individual functions are spread across the system. This means a high cost for the connection or the distribution of the interfaces.

Additionally, there is a rigid chronological pattern in the synchronization of information from the various interfaces (complex synchronization of interfaces). As a result thereof, the chronological pattern is difficult to reproduce or influence when errors occur.

In particular, in a conventional CT system, there are limited opportunities for diagnosis and troubleshooting during commissioning. These limited opportunities for diagnosis and troubleshooting also apply to production. Furthermore, there are also very limited opportunities for diagnosis and troubleshooting when the system is used. Troubleshooting and commissioning initially require a complex configuration of tests.

Furthermore, the rigid structure of the system has the effect that functional enhancements or functional modifications will always affect the function of the other function blocks. For this reason, functional enhancements or functional modifications always require many adaptations of further function blocks.

Furthermore, in the conventional system described, it is difficult to make the entire system observable in order to guarantee an efficient commissioning and error diagnosis. In the conventional system in particular, problems occur in being able to control and monitor the function of individual function blocks and also the interaction of a plurality of function blocks.

A further problem in the structure of the system lies in the fact that the ability to upscale the system by adding further functions, such as, for example, data pre-processing, is only possible at great expense. It also makes it much more difficult to upscale the system by adding further or different detector electronics units in an arrangement such as that shown in FIG. 1.

### SUMMARY

At least one embodiment of the present invention provides a more flexible imaging system that is easier to test.

At least one embodiment of the present invention is directed to an imaging device and/or a method.

The medical imaging device according to at least one embodiment of the invention comprises a central communications unit, at least one individual detector control unit, a plurality of individual detectors, and a serial interface between the at least one individual detector control unit and the central communications unit. For example, the medical imaging device according to the invention can be a computed tomography system. Furthermore, the structure described can also be used in what is known as “molecular imaging”. The apparatus used for this includes a large number of detectors, the measured data for which is transmitted at a high data rate.

In the method according to at least one embodiment of the invention, a central communications unit is arranged between at least one individual detector control unit and a control and image data transmission unit. In addition, a serial interface is embodied between the at least one individual detector control unit and the central communications unit.

The dependent claims and the description that follows contain particularly advantageous further developments and variants of the invention, wherein in particular the claims in one category can be further developed by analogy with the dependent claims in a different claim category.

The imaging device according to at least one embodiment of the invention can further comprise a control and image data transmission unit, which is designed to communicate control and image data with the central communications unit.

In one embodiment of the production method, the individual detector control units can each be identically constructed. The use of identically-constructed units allows the use of a modular architecture, with which a particularly good flexibility and modifiability of the entire system is achieved.

### BRIEF DESCRIPTION OF THE DRAWINGS

The invention will be explained again in further detail below with reference to the attached figures, by means of embodiments. Components that remain the same in the various figures are denoted by identical reference signs. In the figures:

FIG. 1: shows a schematic diagram of a computed tomography system according to the prior art,

FIG. 2: shows a schematic diagram of an embodiment of a device according to a first embodiment of the invention,

FIG. 3: shows a schematic diagram of an embodiment of a device according to a second embodiment of the invention,

FIG. 4: shows a flow diagram of a method for manufacturing a medical imaging system according to an embodiment of the invention.

### DETAILED DESCRIPTION OF THE EXAMPLE EMBODIMENTS

Various example embodiments will now be described more fully with reference to the accompanying drawings in which only some example embodiments are shown. Specific structural and functional details disclosed herein are merely representative for purposes of describing example embodiments. The present invention, however, may be embodied in many alternate forms and should not be construed as limited to only the example embodiments set forth herein.

Accordingly, while example embodiments of the invention are capable of various modifications and alternative forms, embodiments thereof are shown by way of example in the drawings and will herein be described in detail. It should be understood, however, that there is no intent to limit example embodiments of the present invention to the particular forms disclosed. On the contrary, example embodiments are to cover all modifications, equivalents, and alternatives falling within the scope of the invention. Like numbers refer to like elements throughout the description of the figures.

Before discussing example embodiments in more detail, it is noted that some example embodiments are described as processes or methods depicted as flowcharts. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Methods discussed below, some of which are illustrated by the flow charts, may be implemented by hardware, software, firmware, middleware, microcode, hardware description languages, or any combination thereof. When implemented in software, firmware, middleware or microcode, the program code or code segments to perform the necessary tasks will be stored in a machine or computer readable medium such as a storage medium or non-transitory computer readable medium. A processor(s) will perform the necessary tasks.

Specific structural and functional details disclosed herein are merely representative for purposes of describing example embodiments of the present invention. This invention may, however, be embodied in many alternate forms and should not be construed as limited to only the embodiments set forth herein.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items.

It will be understood that when an element is referred to as being “connected,” or “coupled,” to another element, it can be directly connected or coupled to the other element or intervening elements may be present. In contrast, when an element is referred to as being “directly connected,” or “directly coupled,” to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

In the following description, illustrative embodiments may be described with reference to acts and symbolic representations of operations (e.g., in the form of flowcharts) that may be implemented as program modules or functional processes include routines, programs, objects, components, data structures, etc., that perform particular tasks or implement particular abstract data types and may be implemented using existing hardware at existing network elements. Such existing hardware may include one or more Central Processing Units (CPUs), digital signal processors (DSPs), application-specific-integrated-circuits, field programmable gate arrays (FPGAs) computers or the like.

Note also that the software implemented aspects of the example embodiments may be typically encoded on some form of program storage medium or implemented over some type of transmission medium. The program storage medium (e.g., non-transitory storage medium) may be magnetic (e.g., a floppy disk or a hard drive) or optical (e.g., a compact disk read only memory, or “CD ROM”), and may be read only or random access. Similarly, the transmission medium may be twisted wire pairs, coaxial cable, optical fiber, or some other suitable transmission medium known to the art. The example embodiments not limited by these aspects of any given implementation.

It should be borne in mind, however, that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

Spatially relative terms, such as “beneath”, “below”, “lower”, “above”, “upper”, and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, term such as “below” can encompass both an orientation of above and below. The device may be otherwise oriented (rotated 90 degrees or at other orientations) and the spatially relative descriptors used herein are interpreted accordingly.

Although the terms first, second, etc. may be used herein to describe various elements, components, regions, layers and/or sections, it should be understood that these elements, components, regions, layers and/or sections should not be limited by these terms. These terms are used only to distinguish one element, component, region, layer, or section from another region, layer, or section. Thus, a first element, component, region, layer, or section discussed below could be termed a second element, component, region, layer, or section without departing from the teachings of the present invention.

The imaging device according to at least one embodiment of the invention can further comprise a control and image data transmission unit, which is designed to communicate control and image data with the central communications unit.

In one embodiment, the imaging device according to the invention can comprise a plurality of individual detector control units, each being identical in construction.

In one variant of the imaging device according to at least one embodiment of the invention, a serial interface can be arranged in each case between each of the individual detector control units and the central communications unit.

In one specific embodiment, the imaging device can comprise precisely one single control, testing and monitoring channel, which is arranged between the control and image data transmission unit and the central communications unit.

Furthermore, the central communications unit can be configured to combine the data from the individual detector control units.

Furthermore, the central communications unit can be configured to pre-process the measured data supplied by the individual detector control units.

In addition, the central communications unit can be configured to carry out a status evaluation. The status of individual components can therefore be established and evaluated.

The central communications unit can be configured, moreover, to forward control data, test data and image data to the individual detector control units.

Finally, the central communications unit can also be configured to effect the generation of the trigger signal for the individual detectors and to control and carry out the distribution of data to the individual detector control units.

In one embodiment of the production method, the individual detector control units can each be identically constructed. The use of identically-constructed units allows the use of a modular architecture, with which a particularly good flexibility and modifiability of the entire system is achieved.

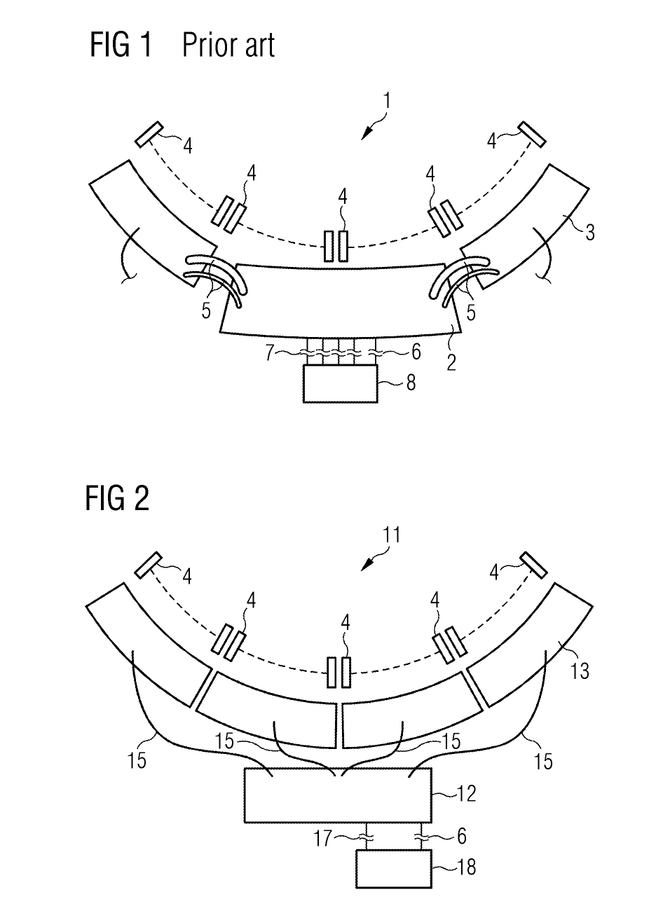

FIG. 1 shows a hardware structure of a conventional detector system 1 divided into three main parts, namely two individual detector control units 3 and a combined individual detector control unit and communications unit 2 with parallel data lines 5 between the individual detector control units 3 and the combined individual detector control unit and communications unit 2. The figure further shows individual detectors 4, which are connected to the individual detector control units 3 and likewise to the combined individual detector control unit and communications unit 2. The combined individual detector control unit and communications unit 2 is connected to a stationary CT unit 8 via a plurality of parallel data lines, that is, four control, testing and monitoring channels, together with a data channel 6.

In the arrangement according to FIG. 1, no data processing takes place on the individual detector control units. The data from the individual detectors are forwarded directly to the combined individual detector control unit and communications unit 2. The combined individual detector control unit and communications unit 2 also includes functions relating to data processing, programming and control of the detector unit.

An extension of the arrangement 1 is only possible with difficulty since the combined individual detector control unit and communications unit 2 is configured only for a specific number of parallel connections and, due already to the dimensions thereof and to the fact that it has to be synchronized with the individual detectors 4 that are connected to it, it cannot be upscaled when required. Consequently, the basic concept underlying the imaging device shown in FIG. 1 is not suitable for an upscaling of the arrangement.

With a conventional imaging system architecture, the control of the data exchange is set for a specific architecture. For example, the data is identified on the basis of the pins to which they fit and are transmitted via separate parallel data transmission channels. If the architecture is intended to be scaled up, it would be necessary to change the entire data transmission structure and the software too. As a result thereof, upscaling an imaging system structured in such a way is impracticable.

In a conventional medical technology imaging system, in a computed tomography system (see FIG. 1), for example, the coding of the data transmitted in the system is usually oriented according to the physical properties of the transmission interface. Status and control information is inserted into the actual data and transmitted through the entire data processing chain or communications chain. Information from various data sources (image data, control information and status information) are combined in one data structure and transmitted in one unit. This transmission in one unit can be visualized as transmission of a data stream.

The information required for commissioning, diagnosis and troubleshooting is extracted from the entire data stream. When the existing data structure is upscaled or changed, the entire system is adapted to the new data structure. There is a rigid connection between the individual data blocks. Consequently only the connections implemented during the system development are available. In the event of modifications or changes to the system, the entire system, the hardware or the software has to be modified anew on all levels. The structure described is therefore too inflexible for frequent modifications or changes.

FIG. 2 illustrates the structure of an imaging device 11 according to an embodiment of the invention. The arrangement includes a central communications unit 12, four individual modular detector control units 13, a plurality of individual detectors 4 and a stationary CT system 18.

In this arrangement, the hardware is divided up such that a serial interface 15 is arranged between the two interfaces to be served, that is, the interface with the individual detectors 4, and the interface with the stationary CT system 18, also known as a control and image data transmission unit. The serial interface 15 can be an HSSL-connection with two differential pairs of a twisted-pair line. This includes a pair for the up-link (triggers, control commands) and a pair for the down-link (status, image data).

Through this, a clear separation is achieved between the function of the individual detector control by the individual detector control units 13, which are now modular in construction, and the function of the image data transmission and control by the central communications unit 12. The CT system 18 is connected via a serial interface for the transmission of control, testing and monitoring data with the central communications unit 12. Furthermore, the central communications unit 12 is connected to the stationary CT system 18 via a data channel 6.

The central communications unit 12 performs various technical functions. This includes, for example, combining the data from the individual detector control units 13 and likewise the data pre-processing and status evaluation. The control data, test data and image data is forwarded to the individual detector control units 13. Moreover, the central communications unit 12 is responsible for the generation of a trigger and distribution of the data to the individual detector control units 13.

The data communication between the individual detector control units 13 and the central communications unit 12 is achieved via a serial interface or a plurality of serial interfaces 15. These interfaces 15 can be, for example, bidirectional serial high-speed data transmission interfaces. In this way, only a simple plug-type connector with few contacts is required between the central communications unit 12 and the individual detector control units 13, which are now modular in construction, and consequently the number of electrical connections from the individual detectors to the central communications unit is reduced. Different functions and components with separate, specific buses and communications interfaces are controlled via a universal protocol.

In the arrangement in FIG. 2, the hardware structure of the detector system 11 is divided into four individual detector-control units 13 and a central communications unit 12 with simple and few connections. The individual detector control units 13 are modular in construction and are connected via serial interfaces 15 with the central communications unit 12. The result achieved by the modular construction or division is that the control of the individual detectors 4 is not divided up over the entire detector system 11, but is restricted in a dedicated manner to the units for the control of the individual detectors 13.

In the event of changes to the interface with the individual detectors 4, only one part of the detector system now has to be exchanged. For example, in the event of a change in the transducer-ASICs of the individual detectors 4, it is now only the control units 13 that have to be changed, even if the function and the control of the new components change considerably. To be more precise, it is now only the interface between the individual detector control units 13 and the individual detectors 4 that has to be changed, while the remaining components, in particular the central communications unit 12 and the data interface 15 between the central communications unit 12 and the individual detector control units 13, can remain unchanged.

Likewise, the protocol for the communication between the central communications unit 12 and the individual detector control units 13 can remain unchanged. Consequently, the adaptability and the cost involved in adapting to a new individual detector 4 is considerably improved or reduced. This is very advantageous in particular in the development of the detectors 4. As a result thereof, this becomes more straightforward and less expensive.

Furthermore, through the reduction in the number of individual detectors 4 controlled per flat panel, that is, per individual detector control unit, the complexity of the flat panels and the size thereof is reduced. Dividing up the individual detector control between a plurality of preferably identical flat panels makes the detector system 11 more transparent and more manageable. Moreover, as a result thereof, there is only one type of flat panel 13, which is used multiple times to control the individual detectors 4.

In addition, the system 11 is simplified considerably by the low number of electrical connectors 15 for serial data transmission. The rigid parallel connectors 5 are replaced by a serial connector 15. This results in reduced circuitry. The modular construction makes it possible for there to be scalability of the hardware system. Furthermore, very different arrangements can be implemented with the same hardware. Finally, a reduction in hardware, logistics, and servicing costs is achieved.

For example, data can be transmitted in a packet-oriented manner via the serial interfaces 15 of the imaging system 11 with a generic data structure. A generic data structure is not specified for one type of signals or data, but allows various data or signals to be transmitted between various function blocks. Advantageously, as a result of the generic data structure, conclusions can be directly drawn regarding the type and allocation of the data.

Unlike the data stream that is conventionally used, the data is therefore no longer identified by means of the position thereof in the data stream. This allows considerably greater flexibility to be introduced when modifying the system. This is because the software no longer has to be painstakingly adapted individually to a change in the hardware since the data are now identifiable via an abstract data structure and no longer via a concrete position in the data stream, which corresponds to a specific fixed hardware arrangement and changes constantly with every modification of the hardware.

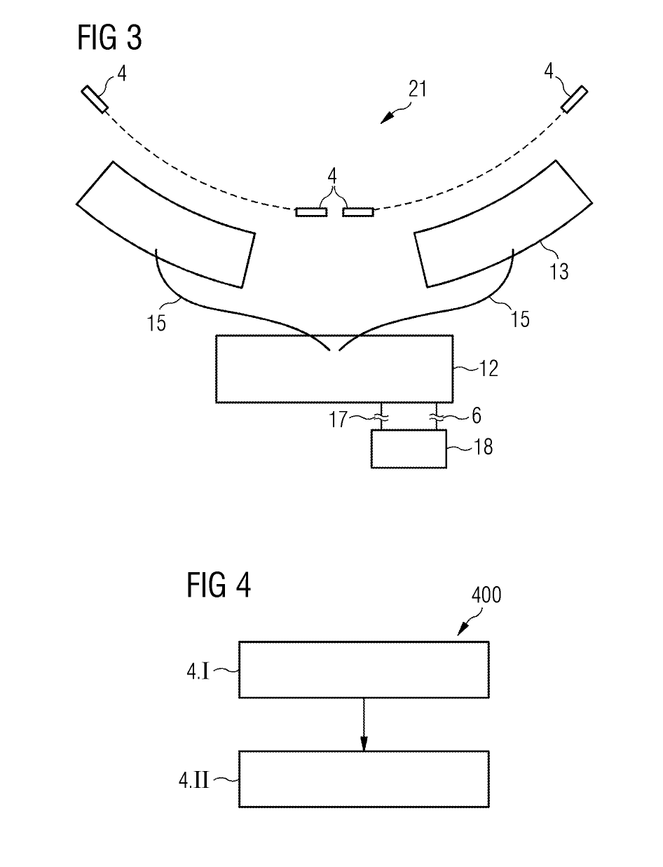

FIG. 3 illustrates an imaging system 21 according to a second embodiment of the invention. The arrangement 21 is constructed in a similar manner to the arrangement 11. It comprises a stationary CT system or a control and image data transmission unit 18, which is connected via a data line 6 and a control-, testing- and monitoring-data transmission line 17 to a central communications unit 12. The central communications unit 12 is connected via serial interfaces 15 to two individual detector control units 13. The individual detector control units 13 are in contact with individual detectors 4.

However, in this embodiment, the individual detectors 4 are arranged or configured differently from the individual detectors in FIGS. 1 and 2. Through the modular construction, a scalability and re-usability of hardware components is achieved. Even with a change in the position or orientation of the individual detectors 4, as shown in FIG. 3, a modification of the arrangement is therefore possible without any problems.

FIG. 4 illustrates a method 400 for manufacturing an imaging device 11. In the method 400, in a step 4.I, a central communications unit 12 is arranged between a plurality of individual detector control units 13 and a control and image data transmission unit 18. Furthermore, in a step 4.II, a serial interface 15 is configured between the individual detector-control units 13 and the central communications unit 12.

In conclusion, it is pointed out once again that the detailed method and structures described in the aforementioned involve embodiments and that the basic principle can even be varied in many respects by a person skilled in the art, without departing from the scope of the invention insofar as it is set out in the claims. It is pointed out in particular that the device according to the invention can be used in various imaging systems. For the sake of completeness, it is also pointed out that the use of the indefinite article “a” or “an” does not preclude the relevant features from also being present a number of times. Likewise the term “unit” or “module” does not preclude this or these from consisting of a plurality of components, which optionally can also be spatially distributed.

The patent claims filed with the application are formulation proposals without prejudice for obtaining more extensive patent protection. The applicant reserves the right to claim even further combinations of features previously disclosed only in the description and/or drawings.

The example embodiment or each example embodiment should not be understood as a restriction of the invention. Rather, numerous variations and modifications are possible in the context of the present disclosure, in particular those variants and combinations which can be inferred by the person skilled in the art with regard to achieving the object for example by combination or modification of individual features or elements or method steps that are described in connection with the general or specific part of the description and are contained in the claims and/or the drawings, and, by way of combinable features, lead to a new subject matter or to new method steps or sequences of method steps, including insofar as they concern production, testing and operating methods.

References back that are used in dependent claims indicate the further embodiment of the subject matter of the main claim by way of the features of the respective dependent claim; they should not be understood as dispensing with obtaining independent protection of the subject matter for the combinations of features in the referred-back dependent claims. Furthermore, with regard to interpreting the claims, where a feature is concretized in more specific detail in a subordinate claim, it should be assumed that such a restriction is not present in the respective preceding claims.

Since the subject matter of the dependent claims in relation to the prior art on the priority date may form separate and independent inventions, the applicant reserves the right to make them the subject matter of independent claims or divisional declarations. They may furthermore also contain independent inventions which have a configuration that is independent of the subject matters of the preceding dependent claims.

Further, elements and/or features of different example embodiments may be combined with each other and/or substituted for each other within the scope of this disclosure and appended claims.

Still further, any one of the above-described and other example features of the present invention may be embodied in the form of an apparatus, method, system, computer program, tangible computer readable medium and tangible computer program product. For example, of the aforementioned methods may be embodied in the form of a system or device, including, but not limited to, any of the structure for performing the methodology illustrated in the drawings.

Even further, any of the aforementioned methods may be embodied in the form of a program. The program may be stored on a tangible computer readable medium and is adapted to perform any one of the aforementioned methods when run on a computer device (a device including a processor). Thus, the tangible storage medium or tangible computer readable medium, is adapted to store information and is adapted to interact with a data processing facility or computer device to execute the program of any of the above mentioned embodiments and/or to perform the method of any of the above mentioned embodiments.

The tangible computer readable medium or tangible storage medium may be a built-in medium installed inside a computer device main body or a removable tangible medium arranged so that it can be separated from the computer device main body. Examples of the built-in tangible medium include, but are not limited to, rewriteable non-volatile memories, such as ROMs and flash memories, and hard disks. Examples of the removable tangible medium include, but are not limited to, optical storage media such as CD-ROMs and DVDs; magneto-optical storage media, such as MOs; magnetism storage media, including but not limited to floppy disks (trademark), cassette tapes, and removable hard disks; media with a built-in rewriteable non-volatile memory, including but not limited to memory cards; and media with a built-in ROM, including but not limited to ROM cassettes; etc. Furthermore, various information regarding stored images, for example, property information, may be stored in any other form, or it may be provided in other ways.

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A medical imaging device, comprising:
a central communications unit;
at least one individual detector control unit;
a plurality of individual detectors; and
a serial interface between the at least one individual detector control unit and the central communications unit.

2. The medical imaging device of claim 1, further comprising:
a control and image data transmission unit, configured to communicate control and image data with the central communications unit.

3. The medical imaging device of claim 1, wherein the at least one individual detector control unit includes a plurality of individual detector control units, each being identically constructed.

4. The medical imaging device of claim 3, wherein the serial interface includes a plurality of serial interfaces and wherein a respective one of the plurality of serial interfaces is arranged in between respective ones of the plurality of individual detector control units and the central communications unit.

5. The medical imaging device of claim 2, further comprising:
precisely one single control, testing and monitoring channel, arranged between the control and image data transmission unit and the central communications unit.

6. The medical imaging device of claim 1, wherein the medical imaging device is a computed tomography system.

7. The medical imaging device of claim 2, wherein the central communications unit is configured to combine measured data from the plurality of individual detector control units.

8. The medical imaging device of claim 2, wherein the central communications unit is configured to pre-process measured data supplied by the plurality of individual detector control units.

9. The medical imaging device of claim 1, wherein the central communications unit is configured to carry out a status evaluation.

10. The medical imaging device of claim 1, wherein the central communications unit is configured to forward control data, test data and image data to the at least one individual detector control unit.

11. The medical imaging device of claim 1, wherein the central communications unit is configured to carry out a generation of a trigger signal for the plurality of individual detectors.

12. The medical imaging device of claim 1, wherein the central communications unit is configured to control and carry out the distribution of data to the plurality of individual detector control units.

13. A method for manufacturing an imaging device, comprising:
arranging a central communications unit between at least one individual detector control unit and a control and image data transmission unit; and
forming of a serial interface between the at least one individual detector control unit and the central communications unit.

14. The method of claim 13, wherein the at least one individual detector control unit includes a plurality of individual detector control units, wherein the central communications unit is arranged between the plurality of individual detector control units and a control and image data transmission unit, and wherein the individual detector control units are identically constructed.

15. The medical imaging device of claim 2, wherein the at least one individual detector control unit includes a plurality of individual detector control units, each being identically constructed.

16. The medical imaging device of claim 15, wherein the serial interface includes a plurality of serial interfaces and wherein a respective one of the plurality of serial interfaces is arranged in between respective ones of the plurality of individual detector control units and the central communications unit.

17. The medical imaging device of claim 16, wherein the medical imaging device is a computed tomography system.
